(19) United States
(12) Patent Application Publication
(10) Pub. No.: US 2020/0214971 A1
(43) Pub. Date: Jul. 9, 2020

(54) HAIR AND SCALP NUTRITION FOR PSORIASIS AND ECZEMA

(71) Applicant: Shirley Coley, Arlington, TX (US)

(72) Inventor: Shirley Coley, Arlington, TX (US)

(21) Appl. No.: 16/731,889

(22) Filed: Dec. 31, 2019

Related U.S. Application Data
(60) Provisional application No. 62/788,250, filed on Jan. 4, 2019.

(51) Int. Cl. A61K 8/9794; A61K 8/02; A61K 8/9789; A61K 8/67; A61K 8/92; A61K 8/64; A61Q 19/00; A61Q 5/00

(52) U.S. Cl. CPC A61K 8/9794; A61K 8/022; A61K 8/9789; A61K 8/67; A61K 2800/805; A61K 8/645; A61Q 19/007; A61Q 5/006; A61K 8/922

(57) ABSTRACT

A compound and method of treating Psoriasis and Eczema includes applying a liquid solution to a part of the body. The solution includes a quantity of natural elements in a mixed powder form stirred into a water solution. Additional elements from natural products are included after the solution rests for a period of time. The method includes applying the solution via a liquid spray to the scalp either before or after shampooing and conditioning occurs.

### CROSS REFERENCE TO RELATED APPLICATIONS

This application claims the benefit of earlier filing date and right of priority to U.S. Provisional Application No. 62/788,250, filed 4 Jan. 2019, the contents of which is incorporated by reference herein in its entirety.

### BACKGROUND OF THE INVENTION

### 1. Field of the Invention

The present application relates generally to a formulated compound, and in particular to a compound used to alleviate symptoms related to Psoriasis and Eczema.

### 2. Description of Related Art

Eczema is a condition where the skin becomes itchy, cracked, rough, and red in selected patches. Various types of eczema is possible. The usual type is “Atopic” where it refers to a collection of diseases relating to the immune system. It may be causes by certain foods, such as nuts and dairy. The symptoms vary according to the age of the person, but frequently include scaly, itchy patches of skin. Other triggers for Eczema are environmental factors like smoke and pollen. Treatment focuses on healing the damaged skin and alleviating symptoms. Presently the goal is to alleviate symptoms of Eczema only as there is no present cure.

Psoriasis is a skin disease that causes itchy or sore patches of thick, red skin with silvery scales. It shows up in certain areas on the body for most of those affected. It is caused by a problem with the immune system. Skin cells that grow deep in the skin rise to the surface. Normally this take some time and occurs gradually over about a month. However, with Psoriasis this occurs in a matter of days as the cells rise too fast. Conditions which contribute to its symptoms include infections, stress, dry skin, and even some medications.

Although great strides have been made with respect to treatments, considerable shortcomings remain.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a chart of a process of making a compound according to an embodiment of the present application.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

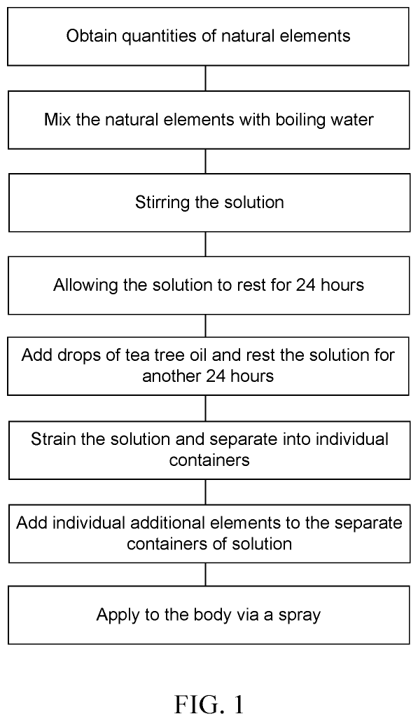

As seen in FIG. 1, the compound and method in accordance with the present application overcomes one or more of the above-discussed problems commonly associated with conventional treatment devices and methods. Specifically, the compound is formulated with natural and organic ingredients. Its purpose is to breakup psoriasis on the scalp, thus removing the scaled patches on the skin. It clears up irritation, itching, and redness thus, improving scalp health. Scaled patches do not reappear as long as the product is used on a regular basis. The product does not contain engineered chemicals. These and other unique features of the device are discussed below and illustrated in the accompanying drawings.

The compound and method will be understood as to its operation, from the accompanying drawings, taken in conjunction with the accompanying description. It should be understood that various ingredient, parts, and features of the compound may be combined together and/or interchanged with one another, all of which are within the scope of the present application, even though not all variations and particular embodiments are shown in the drawings. It should also be understood that the mixing and matching of features, elements, and/or functions is expressly contemplated herein so that one of ordinary skill in the art would appreciate from this disclosure that the features, elements, and/or functions of one embodiment may be incorporated into another embodiment as appropriate, unless otherwise described.

The compound and method of the present application includes a formulation of a compound that includes assorted natural elements combined over a multi-day process. In the first day, 1 rounded tablespoon with equal parts of the following powder mixture is added to a 2-quart container. The powder mixture includes at least some of the following: Barbadensis (aloe Vera), Eclipta alba (bhringraj), Emblica officinalls fruit extract (amla), Nardostachys jatamansi rhizome (jatamansi), Azadirachta indica (neem) Lawsonia inermis (mehendi), Acacia consina (shikakai), Centella asiaticica leaf extract (brahmi), Rosa sinenis flower (hibiscus) and Trigonella foenum-graecum (methi). The 2-quart container is then filled with boiling water to the 2 quart mark. The mixture is stirred, and a lid is placed on top where the mixture is then allowed to sit undisturbed for a 24-hour period.

Next, the container is opened to allow for 3 drops of tea tree oil (a natural preservative) wherein the lid is replaced and the mixture is allowed another 24-hour period to sit. On the third day the liquid mixture is strained into another 2-quart container to filter out the powder residue that did not dissolve. The liquid mixture is then separated into smaller containers. Each smaller container would receive a portion of the liquid mixture followed by a plurality of other ingredients to complete the compound. For example, 12 16-ounce bottles may be used to separate the liquid mixture. In this example, ¾ cup of the liquid mixture is added to each smaller container. Additionally, each smaller container receives the following additional ingredients:
¼ cup of Barbadensis;
1 tablespoon of rose water;
½ teaspoon glycerol (vegetable glycerin);
1.0 ml Biopeiderm (Biotin);
0.5 ml tocopheryl acetate (Vitamin E);
0.5 ml d-panthenol (pro-Vitamin BS);
3 drops of each of the following: Camellia sinensis leaf extract (green tea) extract, Cocodimonium hydroxypropyl hydrolyzed rice protein (rice quat), Centella asiatica (guto kola), Rheum rhabarbarum (rhubarab root) extract, cedarwood extract, Rosmarinus officinalis (rosemary) extract, Rhodiola (Rosea root) extract, Hydrolyzed jojoba protein;
4 drops Melaleuca alternifolia (tea tree) essential oil;
0.5 ml Rhem rhabarbarum (rhubarb root) extract;
¾ cup water; and
½ teaspoon citric acid (lemon extract).

In this condition, each smaller container is complete with the compound mixture of the present application. Naturally it is understood that variations to the above formula are possible without departing from the scope of the present application. The above example is one formulation. Other similar mixtures are possible. Each container may then be sold and used by users for treatment of Psoriasis and Eczema.

Application on the user is preferably done via a wet spray. One application is where the user sprays the resultant mixture on the scalp and hair after the shampoo and condition process. This resultant mixture is allowed to stay on the hair without rinsing. The hair may be combed, detangled, and style as normal. The resultant mixture should be re-applied after each shampoo and conditioning of the hair. The product is safe enough to use daily on dried hair without the need to shampoo or condition.

It should be understood that some slight variations in the compound mixture can result in the mixture producing a cream or lotion. In this manner the application process is similar to other creams where it would be spread out over an affected area. It is also understood that other benefits come through use of the resultant mixture on the scalp. Some of these benefits include the promoting of hair growth and minimizing of dandruff.

It is evident by the foregoing description that the subject application has other significant benefits and advantages. The present compound and method is amenable to various changes and modifications without departing from the spirit thereof. The particular embodiments disclosed above are illustrative only, as the compound and method may be modified and practiced in different but equivalent manners apparent to those skilled in the art having the benefit of the teachings herein. It is therefore evident any alterations, modifications, and all such variations are considered within the scope and spirit of the application. It is apparent that a compound and method with significant advantages has been described and illustrated.

What is claimed is:

1. A compound for treating Psoriasis and Eczema, comprising:
a powder mixture of natural elements including at least some of Barbadensis, Eclipta alba, Emblica officinalls fruit extract, Nardostachys jatamansi rhizome, Azadirachta indica, Lawsonia inermis, Acacia consina, Centella asiaticica leaf extract, Rosa sinenis flower and Trigonella foenum-graecum;
a water solution at a raised temperature at least above its boiling point;
a quantity of tea tree oil mixed in with the powder mixture and water solution; and
additional quantities of natural elements.

2. The compound of claim 1, wherein the ratio of powder mixture to water solution is at least 1 tablespoon to 2 quarts water solution.

3. The compound of claim 1, wherein the quantity of tea tree oil is 3 drops.

4. The compound of claim 1, wherein the tea tree oil is added after the powder mixture is combined with the water solution.

5. The compound of claim 1, wherein the natural elements are at least one of a plant extract, a natural oil, a vitamin, and a plant based protein.

6. A method of treating Psoriasis and Eczema, comprising:
obtaining quantities of natural elements;
mixing the natural elements with a volume of boiling water;
stirring the solution of boiling water and natural elements for a time period;
resting the solution for at least 24 hours;
adding a quantity of tea tree oil into the solution and resting the solution for at least an additional 24 hours;
straining the solution and separating the solution into a plurality of containers;
introducing additional elements into the plurality of containers to create a mixed solution; and
applying the mixed solution.

7. The method of claim 6, wherein applying the mixed solution is done via a wet spray.

8. The method of claim 6, wherein applying the mixed solution is done to a scalp of a user.

9. The method of claim 6, wherein applying the mixed solution is done after shampoo and conditioner are used on the scalp.

10. The method of claim 6, wherein the quantity and type of additional elements added to the solution varies between the plurality of containers.
